(19) United States
(12) Patent Application Publication
(10) Pub. No.: US 2016/0348148 A1
(43) Pub. Date: Dec. 1, 2016

(54) MICROWELL PLATE FOR HIGH-THROUGHPUT DETECTION AND APPLICATION THEREOF

(71) Applicant: SHANGHAI JIAOTONG UNIVERSITY, Shangai (CN)

(72) Inventors: Fang WU, Shanghai (CN); Yueyang ZHOU, Shanghai (CN); Jing YU, Shanghai (CN)

(73) Assignee: SHANGHAI JIAOTONG UNIVERSITY, Shanghai (CN)

(21) Appl. No.: 15/028,522

(22) PCT Filed: Aug. 27, 2014
(86) PCT No.: PCT/CN2014/085244

§ 371 Date: Jul. 29, 2016

(30) Foreign Application Priority Data
Oct. 9, 2013 (CN) 201310467388.1
Oct. 9, 2013 (CN) 201320620517.1

(51) Int. Cl. C12Q 1/527

(52) U.S. Cl. CPC C12Q 1/527; G01N 2333/988; G01N 2500/04; G01N 2500/20

(57) ABSTRACT

A microwell plate for high-throughput detection is provided. The microwell plate includes a plurality of microwell groups, the microwell group includes at least a first microwell and a second microwell, and the microwell group further includes a gas diffusion passage for communicating the first microwell and the second microwell. The application of the microwell plate to high-throughput detection of gas produced through biochemical reaction is also provided. The described methodology is applicable to high-throughput detection and can prevent biological and/or chemical reaction and target substance detection from being interfered with each other, and particularly can attain high-throughput screening of enzyme activity regulators.

### BACKGROUND OF THE PRESENT INVENTION

Field of Invention

The present invention relates to a high-throughput detection device, in particular to a microwell plate applicable to high-throughput detection.

Description of Related Arts

Microwell plate is also usually called as multi-well sample plate and is used for containing multiple (e.g., 6, 12, 24, 48, 96, 384 wells or more) samples. These samples can be detected through various techniques such as autoradiography, liquid scintillation counting and luminescent measurement.

Multi-well microwell plates are mainly used in detection aspects in fields of chemical, biological and pharmaceutical fields to perform analysis and/researches to liquid specimens. A standard microwell plate device usually comprises a microwell plate with a plurality of open wells and a selectable closing device used for closing the wells. Usually, the microwell plate generally comprises a single molded structure. The molded structure comprises a rigid frame for containing the plurality of open wells which are arranged in a rectangular array. Sizes of microwell plates are diversified, the size of each well can be large enough to contain 5 ml of liquid and can be small enough to only contain a little of liquid. In addition, materials of the microwell plates are also diversified and include, for example, polystyrene, polycarbonate, polypropylene, Teflon, glass, ceramic and quartz. Various common traditional microwell plates comprise plates with 96 wells arranged in an 8*12 rectangular array, and plates with more microwells such as plates with 384 wells arranged in a 16*24 rectangular array and plates with 1536 wells arranged in a 32*48 rectangular array.

In a high-throughput detection process, if a target substance can be directly detected in a reaction system, the detection is easier to implement. For example, the target substance is directly detected after reaction by using an enzyme-labeled instrument (Chinese invention patent application titled “Method for quickly screening and evaluating antibacterial bioactive substances”, Application No.: 201010154968.1). If the target substance after reaction cannot be directly detected, for example, detection reagent needs to be added into the reaction system after reaction and detection can be performed by using an instrument after the target substance reacts with the detection reagent, this situation obstructs the progress of high-throughput detection to a certain extent. In the high-throughput detection process, technicians usually face to a greater difficulty that biological and/or chemical reaction and target substance detection cannot be performed in one system. What causes this difficulty includes that the detection reagent will interfere the biological and/or chemical reaction or other substances in the biological and/or chemical reaction will interfere the accuracy and sensitivity of the detection. This difficulty causes that the reaction cannot be performed by means of high-throughput detection and greatly obstructs the progress of development.

Particularly for reaction involving enzyme catalysis, since the catalytic activity of enzyme is easily influenced by the environment, it is very difficult to perform the detection of an enzyme catalysis product in an enzyme catalysis system. For example, H₂S is an important gas signal molecule and has a wide physiological function. H₂S is produced by means of enzyme catalysis mainly through Cystathionine β-synthase (CBS) and Cystathionine γ-lyase (CSE) vitamin B6. Up to now, no high-throughput detection method of high-efficiency and selectivity inhibitors of CBS or CSE has been reported. How to realize high-throughput detection of H₂S gas produced under the catalysis of CBS or CSE is a long-term exiting bottleneck in development of activity regulators thereof, what cause that the high-throughput detection of H₂S gas produced under the catalysis of CBS or CSE cannot be realized are that, if Ellman reagent [5, 5′-dithio bis-(2-nitrobenzoic acid)] (DTNB) for detecting H₂S gas is added into an enzyme catalysis system, enzyme reaction will be interfered and other components in the reaction system will interfere the specificity, accuracy and sensitivity of the detection.

Therefore, one skilled in the art is devoted in developing a technical solution which is applicable to high-throughput detection and can prevent biological and/or chemical reaction and target substance detection from being interfered with each other, and particularly developing a technical solution which can realize high-throughput screening of enzyme activity regulators.

### SUMMARY OF THE PRESENT INVENTION

In view of the above-mentioned defects of the prior art, the technical problem to be solved by the present invention is to provide a microwell plate which is applicable to high-throughput detection and can prevent biological and/or chemical reaction and target substrate detection from being interfered with each other.

In order to achieve the purpose, the present invention provides a microwell plate for high-throughput detection, the microwell plate comprising a plurality of microwell groups, each microwell group comprising at least a first microwell and a second microwell, each microwell group further comprising a gas diffusion passage for communicating the first microwell and the second microwell.

Preferably, the gas diffusion passage is formed in a microwell wall between the first microwell and the second microwell. In the present invention, the microwell wall refers to a portion between the microwells in the microwell plate, i.e., a frame which is used for separating different microwells to enable each microwell to form an independent space.

Further, the first microwell is a reaction well, the second microwell is a detection well and the gas diffusion passage allows at least one reaction product in the first microwell to enter the second microwell through the gas diffusion passage.

Preferably, the gas diffusion passage is a circular, square or a rectangular tubular passage.

Preferably, a distance from an upper edge of the gas diffusion passage to a top of the microwell wall is smaller than two-third of depth of the first microwell.

Preferably, a membrane having a selective permeability effect is arranged in the gas diffusion passage. The membrane having the selective permeability effect refers to a thin film which has selectivity to passage of different particles, i.e., a thin film which only allows a certain type or kind of molecules or particles to enter and exit through diffusion.

Preferably, the microwells in the microwell plate are arranged in a rectangular array, and the first microwells and the second microwells are arranged at spacing.

Further, in order to apply to high-throughput detection of a gas target substrate produced in biochemical reaction, the present invention provides a simple gas diffusion passage arrangement mode, i.e., the gas diffusion passage is a groove formed in the top of the microwell wall. By adopting the arrangement mode, the manufacturing is simple and convenient to perform and the using effect is very good.

Preferably, depth of the groove is smaller than two-third of depth of the first microwell.

More preferably, depth of the groove is smaller than or equal to one-second of depth of the first microwell. In the present invention, with respect to the arrangement depth of the groove, the effect of the present invention can be achieved as long as liquid in the first microwell or the second microwell does not overflow the groove to enter the other side.

Further, the microwell group of the microwell plate for high-throughput detection provided by the present invention further comprises a third microwell, a second passage is formed between the third microwell and the first microwell, and a membrane having a selective permeability effect is arranged in the second passage.

Preferably, the third microwell is a material well, reaction raw material in the third microwell enters the first microwell through the second passage and product after reaction enters the second microwell through the gas diffusion passage.

Purposes of arranging the third microwell in the present invention are that, in some embodiments, cells or microbes can be cultured in the first microwell, the third microwell can provide fresh nutrient substances for these living bodies through the second passage and thus the metabolism demands of the living bodies in the first microwells are satisfied. Metabolism products produced by metabolism of the living bodies in the first microwell can enter the second microwell through the gas diffusion passage such that the metabolism products can be qualitatively or quantitatively detected.

Preferably, the microwell plate provided by the present invention further comprises a sealing device such as a plate sealing film for sealing the microwells.

Further, the present invention provides application of the microwell plate as described above to high-throughput detection of gas produced through biochemical reaction. A biochemical reaction system is prepared in the first microwell and a detection system is prepared in the second microwell. Gas target substance produced through reaction in the first microwells enters the second microwells through the gas diffusion passage, and the target substance is qualitatively or quantitatively detected by means of certain detection such as detection by adopting an enzyme-labeled instruction. Preferably, the gas produced through the biochemical reaction is H₂S, NO, CO, CO₂, C₂H₂, CH₄, O₂, H₂ or NH₃.

The microwell plate provided by the present invention is particularly applicable to screening enzyme activity regulators. An enzyme catalysis system is prepared in the first microwell and comprises an enzyme and reaction substrates thereof, and a detection system is prepared in the second microwell. Candidate compounds to be detected are added into the enzyme catalysis systems in the first microwells, a gas reaction product produced through enzyme catalysis enters the second microwell through the gas diffusion passage, and the enzyme catalysis reaction product is qualitatively or quantitatively detected by means of certain detection such as detection by adopting an enzyme-labeled instruction. Enzyme inhibiting or activating effects of the candidate compounds are evaluated according to detection results of the enzyme catalysis reaction product. In the present invention, the enzyme activity regulators comprise enzyme inhibitors and enzyme activators.

Further, the present invention provides a method for high-throughput screening enzyme activity regulators, wherein specific operations are as follows,
providing a microwell plate which comprising a plurality of microwell groups, each microwell group comprising a reaction microwell and a detection microwell, each microwell group further comprising a gas diffusion passage for communicating the reaction microwell and the detection microwell; and
the method comprises the following steps:
1) biochemical reaction system preparation
preparing a biochemical reaction system in each reaction microwell,
the biochemical reaction system comprising an enzyme, a substrate and candidate compounds;
2) detection system preparation
preparing a detection system in the detection microwell;
3) reaction incubation
sealing the microwell plate by using a plate sealing film to incubate reaction, an enzyme catalysis product produced through reaction in the reaction system entering the detection microwells through the gas diffusion passages and reacting with the detection system; and
4) detection
performing qualitative or quantitative detection to the enzyme catalysis product which enters the detection microwells.

The method for high-throughput screening of enzyme activity regulators provided by the present invention can be implemented by using any one microwell plate provided above.

Further, the method provided by the present invention can be adopted for screening CBS enzyme activity regulators, wherein,
in step 1), the biochemical reaction system comprises Tris-HCl, PLP (Pyridoxal 5′-phosphate), CBS, L-Cys, D,L-HCys, candidate compounds and negative control or positive control;
in step 2) the detection system is DTNB solution;
in step 3), H₂S gas produced through reaction in the reaction system enters the detection microwells through the gas diffusion passages and reacts with DTNB in the detection system; and
in step 4), 413 nM light absorption is determined by using an enzyme-labeled instrument to determine production situations of H₂S gas.

Preferably, while screening CBS enzyme activity regulators, in step 1), in the reaction system, concentration of Tris-HCl is 50 mM, concentration of PLP is 100 μM, concentration of CBS is 100 nM, concentration of L-Cys is 4 mM and concentration of D,L-HCys is 4 mM.

The method is also applicable to detection of H₂S gas produced through CSE catalysis to screen CSE enzyme activity regulators.

Further, the method provided by the present invention can be adopted for screening urease inhibitors, wherein,
in step 1), the biochemical reaction system comprises disodium hydrogen phosphate buffer solution, bovine serum albumin, nickel chloride, urease, urea, candidate compounds, negative control or positive control;
in step 2), the detection system is Nessler's reagent;
in step 3), NH₃ gas produced through reaction in the reaction system enters the second microwells through the gas diffusion passages and reacts with Nessler's reagent in the detection system; and
in step 4), 420 nm light absorption is determined by using an enzyme-labeled instrument to determine production situations of NH₃ gas.

Preferably, while screening CBS enzyme activity regulators, in step 1), in the reaction system, concentration of disodium hydrogen phosphate buffer solution is 50 mM, concentration of bovine serum albumin is 0.025%, concentration of nickel chloride is 100 μM, concentration of urease is 0.0064 U/μl and concentration of urea is 12.5 mM (pH: 7.4; final volume: 50 μL).

In the present invention, the membrane having the selective permeability effect refers to a thin film which only allows a certain type of molecules or particles to enter and exit through diffusion, i.e., a thin film which has selectivity to passage of different particles.

The enzyme activity regulators refer to certain groups which specifically act on enzymes and substances which decrease or increase activity of enzymes, and comprise enzyme inhibitors and enzyme activators.

In the microwell plate provided by the present invention, the microwell is a well with an opening in one end, the opening is located in one surface of a base plate, and one skilled in the art can also call it as a well or hole. Microwells can be circular or square. The microwells described here comprise first microwells and second microwells.

By adopting the microwell plate provided by the present invention, high-throughput screening of enzyme inhibitors can be realized, the detection sensitivity is high and the results are accurate and reliable.

The concept of the present invention, specific structures and technical effects produced thereby will be described below in detail in combination with the drawings, so as to fully understand the purposes, features and effects of the present invention.

### BRIEF DESCRIPTION OF THE DRAWINGS

FIG. 1 is a stereoscopic view of a circular-well microwell plate provided by the present invention.

FIG. 2 is an A-A sectional view of FIG. 1.

FIG. 3 is a microwell plate structure with a membrane having a selective permeability effect.

FIG. 4 is a microwell plate with groove passages provided by the present invention.

FIG. 5 is an A-A sectional view of FIG. 4.

FIG. 6 is a square-well microwell plate with groove passages provided by the present invention.

FIG. 7 is a detection curve chart of a positive compound PPDA in embodiment 6 of the present invention.

### DETAILED DESCRIPTION OF THE PREFERRED EMBODIMENTS

### Embodiment 1

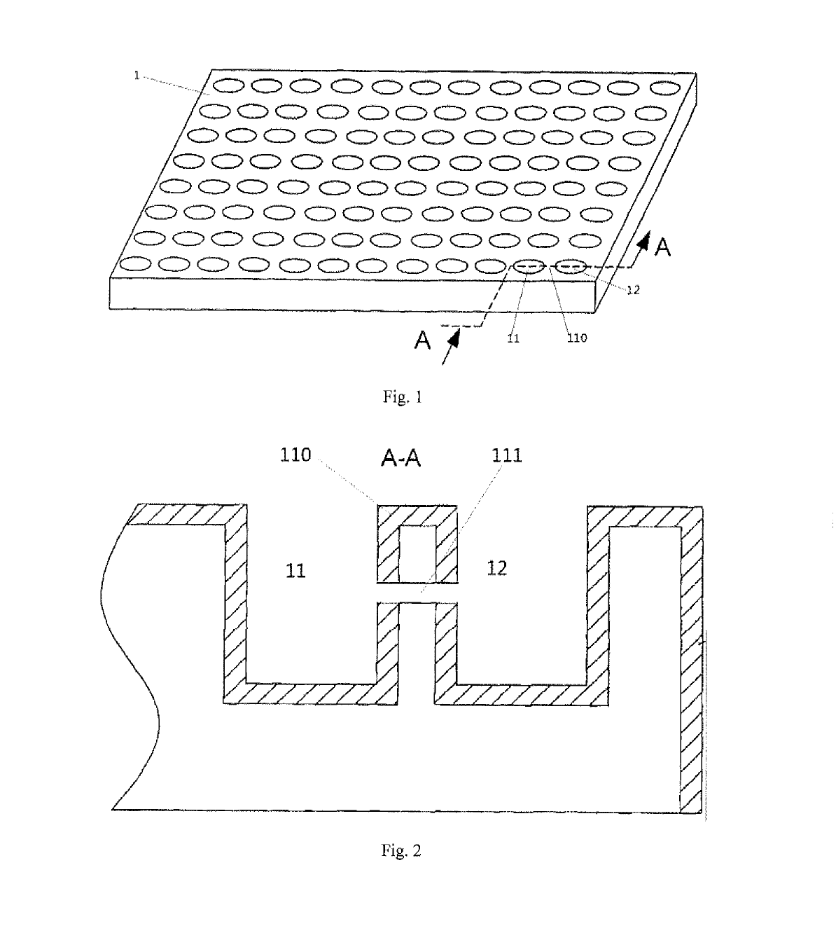

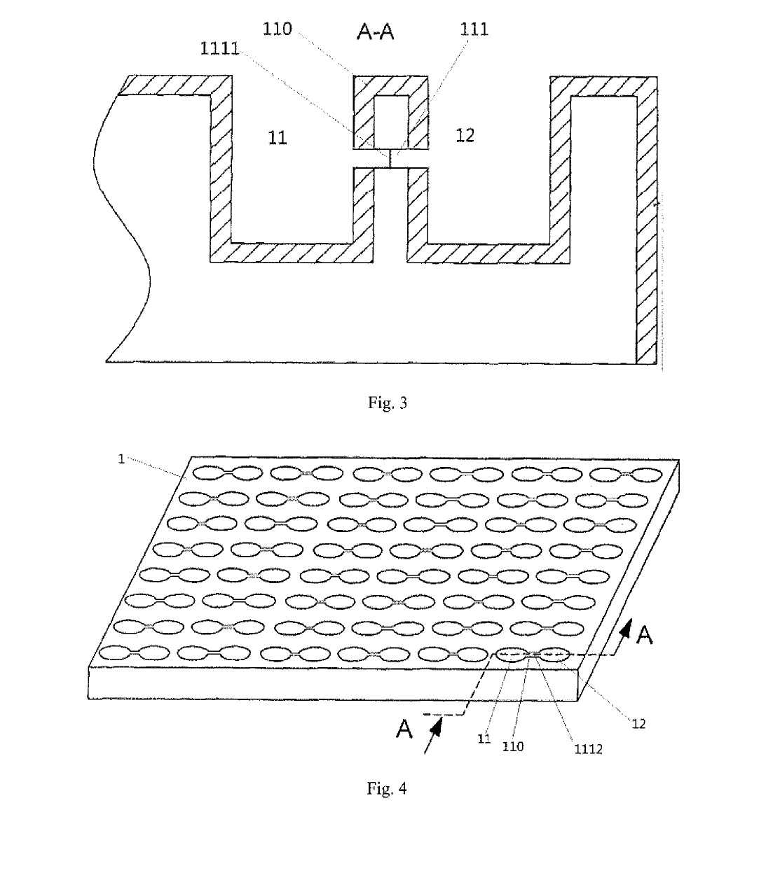

As shown in FIG. 1 and FIG. 2, this embodiment provides a microwell plate for high-throughput detection, which is a plate with 96 wells arranged in an 8*12 rectangular array and comprises a base plate 1 having 48 microwell groups; and each microwell group comprises a first microwell 11 and a second microwell 12, and a gas diffusion passage 111 is formed between the first microwell and the second microwell. Each microwell is a well with an opening in one end and the opening is located in one surface of the base plate 1.

The gas diffusion passage 111 is formed in a microwell wall 110 between the first microwell 11 and the second microwell 12.

The first microwell is a reaction well, the second microwell is a detection well, and during actual use, the gas diffusion passage allows at least one gas reaction product in the first microwell to enter the second microwell through the gas diffusion passage.

The gas diffusion passage can be configured to be a circular, square or a rectangular tubular passage.

A distance from an upper edge of the gas diffusion passage to a top of the microwell wall is smaller than two-third of depth of the first microwell.

### Embodiment 2

The structure of the microwell plate in this embodiment is substantially consistent with the structure of the microwell in embodiment 1 and only has a difference that a membrane 1111 having a selective permeability effect is arranged in the gas diffusion passage 111.

### Embodiment 3

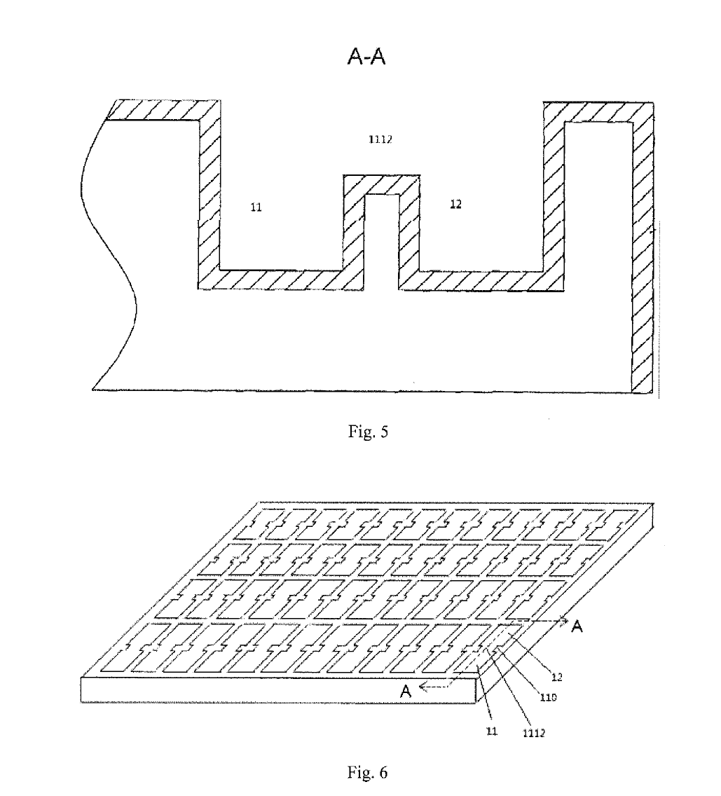

In order to apply to high-throughput detection of a gas target substance produced in biochemical reaction, the microwell plate provided by this embodiment is a plate with 96 wells arranged in an 8*12 rectangular array and comprises 48 microwell groups. This embodiment provides a simple gas diffusion passage arrangement mode. As shown in FIG. 4 and FIG. 5, the gas diffusion passage is a groove 1112 famed in a top of the microwell wall. In this embodiment, depth of the groove is one-second of depth of the first microwell 11. In other implementation modes, the depth of the groove can be arranged according to actual situations, for example, proper depth of groove can be selected according to a volume of liquid in a biochemical reaction system and a detection system, and thus not only can the liquid in the two systems be prevented from being mixed through the groove, but also the gas produced through reaction in the first microwell can be diffused into the second microwell in time.

### Embodiment 4

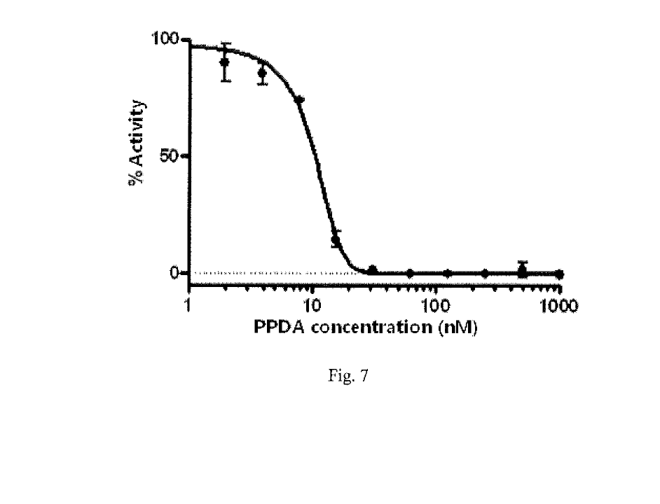

This embodiment provides a square-well microwell plate. As shown in FIG. 6, except the shape of the microwells is different from the shape of the microwells in embodiment 3, the structure of the microwell plate is fully the same as the microwell plate in embodiment 3.

### Embodiment 5

This embodiment provides a specific implementation mode of high-throughput screening of CBS enzyme activity regulators to describe that high-throughput detection of substances produced during biochemical reaction can be realized by using the microwell plate provided by the present invention and thus the high-throughput screening of the enzyme activity regulators can be realized.

Materials used in this embodiment are as follows:

Source of Cell

HepG2 cell refers to a human liver carcinoma cell line purchased from Cell Resource Center of Shanghai Institutes for Biological Sciences, CAS.

Major reagent

| | |
|---|---|
| L-cysteine | Sangon |
| Ellman reagent (DTNB for short) | Sangon |
| D,L-homocysteine | TCI Shanghai |
| L-Cys | Sangon |
| Pig heart malate dehydrogenase | Amresco |
| 1X non-essential amino acid | Life Technologies |
| Fetal bovine serum (10%) | Life Technologies |
| Penicillin (1%, weight/volume) | Life Technologies |
| Streptomycin (1%, weight/volume) | Life Technologies |
| MEM | Life Technologies |

Other chemicals or reagent are purchased from Sigma-Aldrich.

Abbreviations used in the present invention are as follows:
hCBS refers to human cystathionine β-synthase;
hDDC refers to human dopa decarboxylase;
DTNB refers to 5,5′-dithio bis-(2-nitrobenzoic acid);
Tris-HCI refers to Tris(hydroxymethyl)aminomethane hydrochloride;
EDTA refers to Ethylene Diamine Tetraacetic Acid;
L-Cys refers to L-cysteine;
D,L-HCys refers to D,L-homocysteine.
SAM refers to S-adenosyl methionine;
PLP refers to pyridoxal phosphate;
HepG2 cell refers to a human liver carcinoma cell line.

The method adopted in this embodiment is as follows:

(I) Preparation of CBS Enzyme

In this embodiment, the human CBS (hCBS) is adopted and prepared according to a method described in Arch Biochem Biophys 470, 64-72 (2008), Oliveriusova, J., J Biol Chem 277, 48386-94 (2002) by Frank. N., or Acta Crystallogr D Biol Crystallogr 57, 289-91 (2001) by Janosik, M. et al.

(III) Preparation of Human Dopa Decarboxylase (hDDC)

The hDDC enzyme is prepared according to a method described in J Biol Chem 271, 23954-9 (1996) by Bertoldi, M., J Inherit Metab Dis 34, 1213-24 (2011) by Montioli, R., or Plant Cell Physiol 43, 159-69 (2002) by Chen, L. M.

(IV) High-Throughput Screening of hCBS Enzymatic Reaction Inhibitors

Firstly 50 mM Tris-HCl, 100 μM PLP, 100 nM hCBS, 4 mM L-Cys and 4 mM D,L-HCys (pH: 8.6; final volume: 50 μL) were sequentially added into each first microwell (reaction well) of the microwell plate provided in embodiment 3 by using a liquid multi-channel electronic pipette (Thermo Fisher, USA), and then substances to be tested (20 μM or 100 μM) and negative control DMSO (volume fraction: 2%, final concentration) or positive control (20 μM HA, Hydroxylamine, final concentration) were respectively added into corresponding reaction wells. Firstly 50 μL of DTNB (300 μM in 262 mM of Tris-HCl, containing 13 mM of EDTA; pH=8.9) was added into the second microwells (detection wells) of the microwell plate, then the microwell plate was sealed by using an UltraClear plate sealing film (Platemax PCR-TS, USA), incubation was performed for 60 min at 37° C. and 413 nM light absorption was determined by using an enzyme-labeled instrument (Synergy 2).

Totally about 7200 small-molecular compounds were screened, finally 9 CBS enzyme inhibitors were obtained through screening, IC₅₀ thereof was smaller than 50 μM and they were high-efficiency CBS inhibitors.

(V) Screening of Specific Inhibitors of H₂S Signal Transduction Pathways

Since the specificity of the current known hCBS inhibitors was not good, the inhibitors also inhibited other PLP-dependent enzymes, DDC is another type of PLP-dependent enzyme and it was not related to H₂S signal transduction pathways, we further tested the specificity of selected 9 compounds to hCBS.

By referring to the experiment method in Assay Drug Dev Technol. 2010 April; 8(2):175-185 by David C. Smithson, IC₅₀ of the 9 compounds to hDDC was determined. Results are as shown in Table 1.

By comparing the results in Table 1, 4 high-efficiency and high-specificity hCBS inhibitors were screened. These four compounds had selectivity greater than 8 times between hCBS and hDDC (Table 1). Since other two conjugate enzymes, i.e., malate dehydrogenase and pyruvate carboxylase were contained in enzyme reaction liquid during determination of hDDC activity, it indicated that these inhibitors did not influence the activity of the other two enzymes.

TABLE 1

| Compound | IC₅₀ to hCBS (μM) | IC₅₀ to hDDC (μM) |
|---|---|---|
| 1 | 4.0 | 35.0 |
| 2 | 8.0 | 16.0 |
| 3 | 15.0 | 25.0 |
| 4 | 20.0 | >400.0 |
| 5 | 20.0 | 300.0 |
| 6 | 30.0 | 110.0 |
| 7 | 40.0 | 60.0 |
| 8 | 35.0 | 150.0 |
| 9 | 12.0 | 400.0 |

Structures of the compounds are shown as follows:

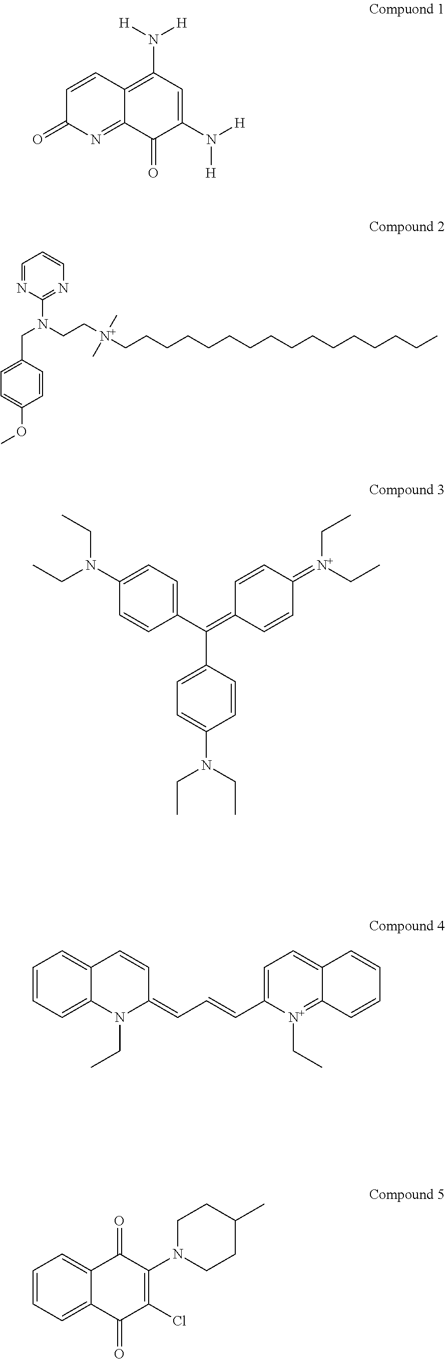

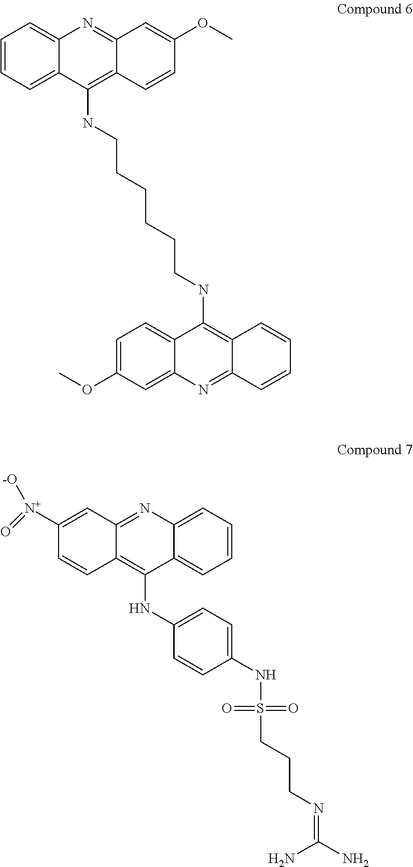

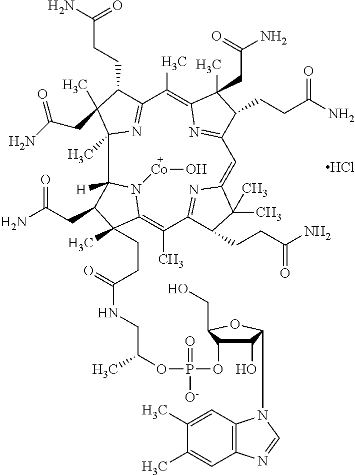

Compound 8 (English name of compound 8 is Hydroxocobalamin, Cas NO. 13422-51-0)

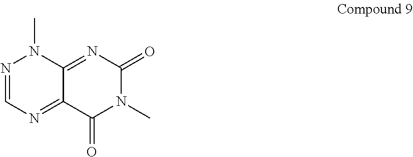

### Embodiment 6

This embodiment provides a specific implementation mode of high-throughput screening urease inhibitors to describe that high-throughput detection of substances produced during biochemical reaction can be realized by using the microwell plate provided by the present invention and thus the high-throughput screening of the enzyme activity regulators can be realized.

In this embodiment, an experiment method adopted for detection of ammonia gas was as follow:

(I) Production and Detection Principle of Ammonia Gas

Urease can specifically catalyze urea hydrolysis to release ammonia and carbon dioxide, the produced ammonia gas can specifically react with Nessler's reagent and a product thereof can be detected at 420 nm.

(II) Source of Urease

In this embodiment, urease from Canavalia ensiformis (Jack bean) was adopted and purchased from Sigma-Aldrich (China) reagent Company, Article Number: U1500.

(III) Nessler's Reagent

Nessler's reagent is reagent which can determine content of ammonia and nitrogen in air or water by using a principle of spectrophotometry and was purchased from Tianjin Tianyi Detection Reagent Shop, Article Number: G500.

(IV) High-Throughput Screening of Urease Inhibitors

Firstly 50 mM of disodium hydrogen phosphate buffer solution, 0.025% bovine serum albumin, 100 μM nickel chloride, 0.0064 U/μL urease and 12.5 mM urea (pH: 7.4; final volume: 50 μL) were sequentially added into each first microwell (reaction well) of the microwell plate provided in the embodiment by using a liquid multi-channel electronic pipette (Thermo Fisher, USA), and then substances to be tested (100 μM) and negative control DMSO (volume fraction: 2%, final concentration) or positive control (10 μM Phenyl phosphorodiamidate, PPDA, final concentration) were respectively added into corresponding reaction wells. Firstly 504 of Nessler's reagent was added into the second microwells (detection wells) of the microwell plate, then the microwell plate was sealed by using an UltraClear plate sealing film (Platemax PCR-TS, USA), incubation was performed for 15 min at 37° C. and 420 nM light absorption was determined by using an enzyme-labeled instrument (Synergy 2). Therein, a detection curve chart of positive compound PPDA is as shown in FIG. 7.

Totally about 1563 small-molecular compounds were screened, finally 3 urease inhibitors were obtained through screening, IC₅₀ of the 3 compounds to urease was determined and was smaller than SO₄M, and specific results are as shown in Table 2.

TABLE 2

| Compound | IC₅₀ to urease (μM) |
|---|---|
| 1 | 10 |
| 2 | 8 |
| 3 | 1 |

Structures of the compounds are shown as follows:

Compound 1′ (English name of compound 1′ is Thonzonium Bromide, Article Number of SIGMA: T7783)

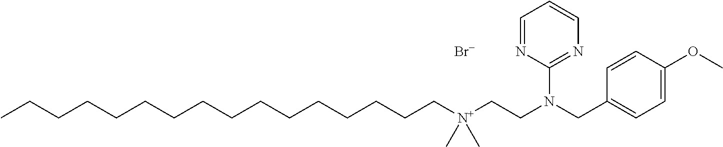 [SMILES: CCCCCCCCCCCCCCCC[N+](C)(C)CCN(Cc1ccc(OC)cc1)c1ncccn1 ; [Br-]]

Compound 2′ (English name of compound 2′ is Quinaldine Blue, Article Number of SIGMA: 166510)

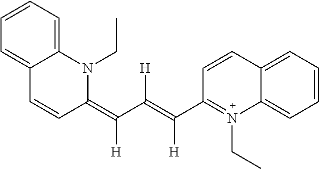

### Compound 3′

CuSO₄

The preferred specific embodiments of the present invention are described above in detail. It should be understood that one skilled in the art can make various modifications and variations according to the concept of the present invention without contributing any inventive labor, Therefore, all technical solutions which can be obtained by one skilled in the art according to the concept of the present invention through logic analysis, reasoning or limited experiments on the basis of the prior art shall also be included in the protection range defined by the claims.

1. A microwell plate for high-throughput detection, the microwell plate comprising a plurality of microwell groups, the microwell group comprising at least a first microwell and a second microwell, the microwell group further comprising a gas diffusion passage for communicating the first microwell and the second microwell; wherein the second micro-well is a detection well and the gas diffusion passage allows at least one gas reaction product in the first microwell to enter the second microwell through the gas diffusion passage.

2. The microwell plate according to claim 1, wherein the gas diffusion passage is formed in a microwell wall of the first microwell and the second microwell.

3. (canceled)

4. The microwell plate according to claim 2, wherein the gas diffusion passage is a circular, square or a rectangular tubular passage.

5. The microwell plate according to claim 4, wherein a distance from an upper edge of the gas diffusion passage to a top of the microwell wall is smaller than two-third of depth of the first microwell.

6. The microwell plate according to claim 2, wherein a membrane having a selective permeability effect is arranged in the gas diffusion passage.

7. The microwell plate according to claim 2, wherein the gas diffusion passage is a groove formed in the top of the microwell wall.

8. The microwell plate according to claim 7, wherein depth of the groove is smaller than two-third of depth of the first microwell.

9. The microwell plate according to claim 7, wherein depth of the groove is smaller than or equal to one-second of depth of the first microwell.

10. The microwell plate according to claim 1, wherein each microwell group further comprises a third microwell, a second passage is formed between the third microwell and the first microwell, and a membrane having a selective permeability effect is arranged in the second passage.

11. The microwell plate according to claim 10, wherein the third microwell is a material well, a reaction raw material in the third microwell enters the first microwell through the second passage and a gas product after reaction enters the second microwell through the gas diffusion passage.

12. The microwell plate according to claim 1, wherein the microwell plate further comprises a sealing device for sealing the microwells.

13. Application of the microwell plate according to claim 1 to high-throughput detection of gas produced through biochemical reaction.

14. The application according to claim 13, wherein the gas is H₂S, NO, CO, CO₂, C₂H₂, CH₄, O₂, H₂ or NH₃.

15. Application of the microwell plate according to claim 1 to high-throughput screening of enzyme activity regulators.

16. The application according to claim 15, wherein the enzyme activity regulators are enzyme inhibitors or enzyme activators.

17. A method for high-throughput screening of enzyme activity regulators, characterized in that,
Providing a microwell plate, the microwell plate comprising a plurality of microwell groups, the microwell group comprising a reaction microwell and a detection microwell, the microwell group further comprising a gas diffusion passage for communicating the reaction microwell and the detection microwell; and
the method comprises the following steps:
1) biochemical reaction system preparation
preparing a biochemical reaction system in each reaction microwell,
the biochemical reaction system comprising an enzyme, a substrate and candidate compounds;
2) detection system preparation
preparing a detection system in each detection microwell;
3) reaction incubation
sealing the microwell plate by using a plate sealing film to incubate reaction, an enzyme catalysis product produced through reaction in the reaction system entering the detection microwells through the gas diffusion passages and reacting with the detection system; and
4) detection
performing qualitative or quantitative detection to the enzyme catalysis product which enters the detection microwells.

18. The method according to claim 17, being adopted for screening CBS enzyme activity regulators, wherein, in step 1), the biochemical reaction system comprises Tris-HCl, PLP, CBS, L-Cys, D,L-HCys and candidate compounds;
in step 2) the detection system is DTNB solution;
in step 3), H₂S gas produced through reaction in the reaction system enters the detection microwells through the gas diffusion passages and reacts with DTNB in the detection system; and
in step 4), 413 nm light absorption is determined by using an enzyme-labeled instrument to determine production situations of H₂S gas.

19. The method according to claim 18, wherein, in step 1), in the reaction system, concentration of Tris-HCl is 50 mM, concentration of PLP is 100 μM, concentration of CBS is 100 nM, concentration of L-Cys is 4 mM and concentration of D,L-HCys is 4 mM.

20. The method according to claim 17, being adopted for screening urease inhibitors, wherein,
in step 1), the biochemical reaction system comprises disodium hydrogen phosphate buffer solution, bovine serum albumin, nickel chloride, urease, urea, candidate compounds, negative control or positive control;
in step 2), the detection system is Nessler's reagent;
in step 3), NH₃ gas produced through reaction in the reaction system enters the second microwells through the gas diffusion passages and reacts with Nessler's reagent in the detection system; and
in step 4), 420 nm light absorption is determined by using an enzyme-labeled instrument to determine production situations of NH₃ gas.

21. The method according to claim 20, wherein, in step 1), in the reaction system, concentration of disodium hydrogen phosphate buffer solution is 50 mM, concentration of bovine serum albumin is 0.025%, concentration of nickel chloride is 100 μM, concentration of urease is 0.0064 U/μL, concentration of urea is 12.5 mM, and pH thereof is 7.4.
